(19) United States
(12) Patent Application Publication
(10) Pub. No.: US 2014/0046263 A1
(43) Pub. Date: Feb. 13, 2014

(54) PROTECTIVE SHOWER GUARD FOR INDWELLING MEDICAL CATHETERS

(71) Applicant: LARRY SALAZAR, Friendswood, TX (US)

(72) Inventor: LARRY SALAZAR, Friendswood, TX (US)

(21) Appl. No.: 13/569,841

(22) Filed: Aug. 8, 2012

(51) Int. Cl. A61M 25/02

(52) U.S. Cl. 604/174

(57) ABSTRACT

A novel shower guard for use by a patient having an indwelling catheter is disclosed. The novel shower guard fits over a standard medical dressing and does not require medical staff to apply, but instead may be affixed over the medical catheter by the patient. Once applied, the patient is able to shower without worrying that the incision will become infected. The shower guard is intended as a single-use device, with the patient removing and disposing of the shower guard following use. The shower guard may be used by kidney dialysis patients, chemotherapy patients, and other patients who use temporary or indwelling medical catheters.

### TECHNICAL FIELD

This application relates to medical catheters and, more particularly, to a disposable medical product useful to patients with temporary or indwelling catheters.

### BACKGROUND

Among other purposes, the kidneys in the human body operate to produce urine. In healthy individuals, this urine production is designed to remove waste from the human body. When the kidneys are not functioning properly, known as renal failure, the individual may have chronic kidney disease (CKD) that has progessed to end stage renal disease (ESRD). Such individuals must seek medical treatment or perish.

Individuals with ESRD have some difficult, but critical, options for resolving their condition. A renal transplant in which the failing kidney is replaced with a healthy kidney, is one option. In lieu of a renal transplant, though, the patient with non-working kidneys is subjected to regularly scheduled dialysis.

Dialysis is the process of removing waste from the blood and thus provides an artificial substitute for functioning kidneys. In addition to ESRD patients, dialysis may be useful to individuals with an acute kidney injury. There are two types of dialysis commonly in use for those with kidney disease, peritoneal dialysis and hemodialysis.

The dialysis patient is fitted with a medical catheter. A medical catheter is a tube with an attached needle that may be inserted into the body of the patient, allowing for vascular access. After sterilizing an appropriate location on the body (which may vary), an incision is made and the medical catheter is inserted at the site of incision. The connected needle is inserted into a vein and the patient's skin is stitched so as to securely couple the medical catheter to the patient. The medical catheter thus gives the medical professional “access” to the inside of the patient's body. For kidney dialysis patients, the medical catheter is sewn into the patient by stitching, and is thus a temporary or permanent catheter, known as an indwelling catheter.

Other individuals besides CKD patients may be fitted with a medical catheter. For example, patients seeking treatment for cancer may be fitted with a medical catheter in order to deliver chemotherapy medicines into the body. Chemotherapy is a cancer treatment in which chemicals are delivered into the body through the medical catheter connected to an intravenous drip bag, to prevent cancer cells within the body from growing and dividing. Medical catheter may be used for patients with multiple sclerosis, fluid buildup in the body, or for a variety of other conditions.

For the patient with kidney failure, upon inserting the medical catheter, the technician or doctor is able to connect the bloodstream of the patient to the dialysis machine. Blood flows from the patient through the medical catheter to the dialysis machine, which then filters out waste and water from the patient's bloodstream, before returning the filtered blood to the patient's body. The single medical catheter is used both for egress of the patients' blood to the dialysis machine for filtration, and for ingress of the filtered blood back to the patient's body. (It is possible to have two medical catheters, one for egress of tainted blood from the patient and the other for ingress of filtered blood to the patient.) Thus, although a simple device, the medical catheter is a critical component in the successful dialysis of the patient. One type of medical catheter is known as a PICC line or PIC line, which is short for peripherally inserted central catheter line.

When the medical catheter is inserted into the patient's body, the technician or nurse covers the device with a medical dressing, such as a gauze bandage or other dressing. The medical dressing is needed to protect the site of insertion from getting dirty or wet, as either may cause an infection. Thus, during the period of time that the medical catheter is in use, patients cannot shower, bathe, swim, or otherwise expose themselves to water.

The average person has ten to twelve pints of blood in her circulatory system at a time. Kidney dialysis patients also experience weight gain due to excess fluid being in the body. Thus, during dialysis, the excess fluid is removed and the blood is filtered. Only about one pint of blood is “missing” from the patient's body during dialysis. Typical treatment for the patient is four hours on the dialysis machine, three times a week, for a total of twelve hours per week. For those patients who are fortunate enough to do home dialysis, overnight dialysis may be performed while the patient is sleeping.

In any case, the medical catheter is an indwelling catheter, and the patient is unable to shower or otherwise get wet while the catheter is inserted. Instructing the patient to elevate the medical catheter while bathing, for example, may be difficult particularly for chronically ill patients, which may increase pain and frustration for them. Because the patients are able to go home between treatments, their activities are not monitored or controlled by medical staff, and the inability to shower between treatments is likely frustrating. Instructions not to shower may go unheeded and the site of incision may become wet, increasing the likelihood of infection.

While being concerned with the patient's comfort, the medical staff is also trying to protect the patient. Each year, hospitals incur thousands of dollars in costs due to patient infection, some of which result in loss of life. The incision in the patient's body may attract bacteria and other infectious agents. It is therefore very important that the site of incision be kept clean and dry.

Products exist to either protect the insertion point or to prevent the insertion point (and medical dressing) from getting wet. Unfortunately, these products are complicated and require manipulation of the medical catheter itself, and thus may only be used by medical professionals. Such solutions are thus not practical for the at-home patient who desires the ability to shower or bathe independently.

Thus, there is a need for a medical device that overcomes the shortcomings of the prior art.

### BRIEF DESCRIPTION OF THE DRAWINGS

The foregoing aspects and many of the attendant advantages of this document will become more readily appreciated as the same becomes better understood by reference to the following detailed description, when taken in conjunction with the accompanying drawings, wherein like reference numerals refer to like parts throughout the various views, unless otherwise specified.

FIG. 1 is a diagram of a shower guard for use with an indwelling medical catheter, according to some embodiments;

FIG. 2 is a diagram of the shower guard of FIG. 1 with dimensions, according to some embodiments;

FIG. 3 is diagram of an indwelling medical catheter inserted into the intrajugular vein of a human body, according to the prior art;

FIG. 4 is a diagram of the indwelling medical catheter, without medical dressing, extending from the insertion site into the body, according to the prior art;

FIG. 5 is a diagram of the indwelling medical catheter with a standard medical bandage covering the insertion site, according to the prior art;

FIG. 6 is a diagram of the shower guard of FIG. 1 being disposed over the indwelling medical catheter with the standard medical bandage of FIG. 5, according to some embodiments;

FIG. 7 is a side view of the shower guard of FIG. 1 showing the peelaway backing disposed over the adhesive, according to some embodiments; and

FIG. 8 is a simplified flow diagram showing how a patient uses the shower guard of FIG. 1, according to some embodiments.

### DETAILED DESCRIPTION

In accordance with the embodiments described herein, a novel shower guard for an indwelling catheter is disclosed. The novel shower guard fits over a standard medical dressing and does not require medical staff to apply, but instead may be affixed over the medical catheter by the patient. Once applied, the patient is able to shower without worrying that the incision will become infected. The shower guard is intended as a single-use device, with the patient removing and disposing of the shower guard following use. The shower guard may be used by kidney dialysis patients, chemotherapy patients, and other patients who use temporary or indwelling medical catheters.

In the following detailed description, reference is made to the accompanying drawings, which show by way of illustration specific embodiments in which the subject matter described herein may be practiced. However, it is to be understood that other embodiments will become apparent to those of ordinary skill in the art upon reading this disclosure. The following detailed description is, therefore, not to be construed in a limiting sense, as the scope of the subject matter is defined by the claims.

For ease of description, the novel shower guard is discussed herein in the context of use for patients with kidney failure such as chronic kidney disease (CKD), and end stage renal disease (ESRD). However, the principles described herein are not meant to limit the use of the novel shower guard merely to uses related to kindey failure, but may be applied to other medical procedures involving indwelling catheters, including, but not limited to, uses for chemotherapy, multiple sclerosis, arthritis, and other illnesses. Further, the novel shower guard may be used with patients who do not suffer from chronic illnesses, but who are temporarily under treatment for impact injuries or other conditions that do not require lifelong treatment and are thus fitted with a temporary catheter.

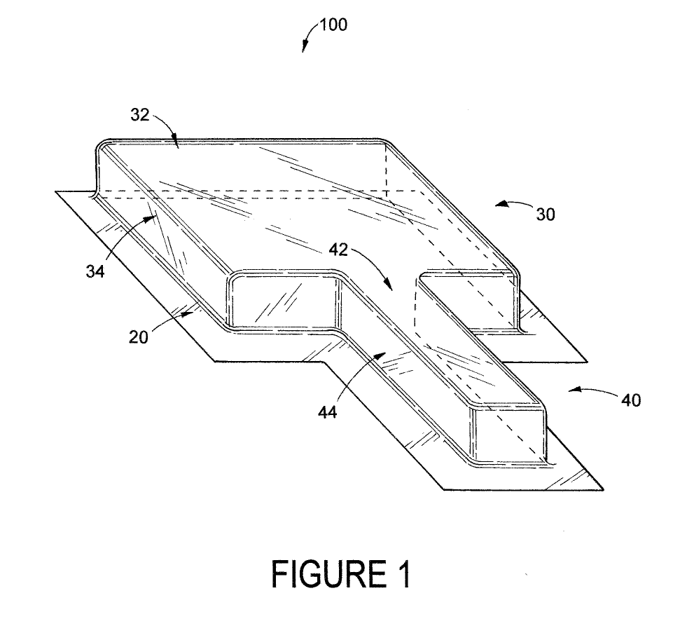

FIG. 1 is a simplified drawing of a novel shower guard 100 for use with a medical catheter, according to some embodiments. The shower guard 100 consists of three parts: an adhesive portion 20, a main portion 30, and an extension portion 40. The main portion 30 includes a first surface 32 and multiple sides 34, where the multiple sides 34 are orthogonal to the surface 32, such that the surface 32 and sides form part of an open rectangular cube. Similarly, the extension portion 40 includes a second surface 44 and multiple sides 44, such that the multiple sides 44 are orthogonal to the surface 44 and form part of a second open rectangular cube. In some embodiments, the surface 32 of the main portion 30 and the surface 42 of the extension portion 40 form a continuous smooth surface. The surfaces 32 and 42 are disposed substantially parallel to the patient's skin while the shower guard 100 is in place.

The adhesive portion 20 is substantially parallel to the surfaces 32 and 42 and is therefore substantially orthogonal to the sides 34 and 44. The adhesive portion 20 connects to the base of each side 34, 44 (e.g., the part to be closest to the patient's skin), surrounds the main portion 30 and the extension portion 40, and extends outward from the main and extension portions so as to be visible around all sides of the shower guard 100. The adhesive portion 20 is tightly coupled to the sides 34, 44 of the main and extension portions 30, 40, such that no air or water may collect therebetween.

The adhesive portion 20 has a top surface and a bottom surface, with the top surface being visible when the shower guard 100 is in place, that is, affixed to the patient, and the bottom surface containing the adhesive backing material. The adhesive portion 20 is to be disposed flat against the patient's body, while the main portion 30 and the extension portion 40 extend orthogonally upward from the patient's body in order to cover up the attached medical catheter hardware (not shown).

In some embodiments, the adhesive portion 20 of the shower guard 100 is made of an elastomeric material (ideally, a non-latex material). The bottom surface (e.g., the surface to be placed against the patient's skin) of the adhesive portion 20 contains an adhesive backing (not shown). In some embodiments, the adhesive backing is moisture-resistant and is continuously disposed upon the bottom surface of the adhesive portion 20. In some embodiments, the adhesive backing is sufficiently agglutinative to allow the shower guard 100 to easily adhere to the skin of the patient. Once the adhesive backing affixes the shower guard 100 to the skin of the patient, a protective seal is formed, preventing water from getting to the catheter hardware, the medical bandage, and, most importantly, the insertion site of the catheter.

In some embodiments, the shower guard 100 is composed of a latex-free, fluid-impermeable material. The main portion 30 and extension portion 40 of the shower guard 100 are made of a material that does not easily degrade upon limited exposure to water. In some embodiments, the main portion 30 and the extension portion 40 are made of a see-through material. The shower guard 100 may thus be composed of a polymer material, a composite material composed of different materials in combination, an elastomeric material, and a myriad of other materials. The main portion 30 and extension portion 40 of the shower guard 100 are rigid enough to maintain their original shape, despite being exposed to water during showering by the patient. In some embodiments, when the novel shower guard 100 is in place over the medical catheter, the guard does not make contact with the catheter hardware.

In some embodiments, the main portion 30 of the shower guard 100 is wide enough that, when removed from the skin of the patient's body after use, the medical bandage remains intact, affixed to the patient, and covering up the incision site. Ideally, neither the medical bandage nor the catheter hardware are touched by the patient when using the shower guard 100.

Another way to view the shower guard 100 is as a pair of open-ended rectangular cubes, with one rectangular cube (the main portion 30) having a cutaway in one of its sides so as to fittedly couple to the second rectangular cube (the extension portion 40). The two rectangular cubes are entirely opened on one side so that the shower guard 100 may cover or house the medical bandage and medical catheter.

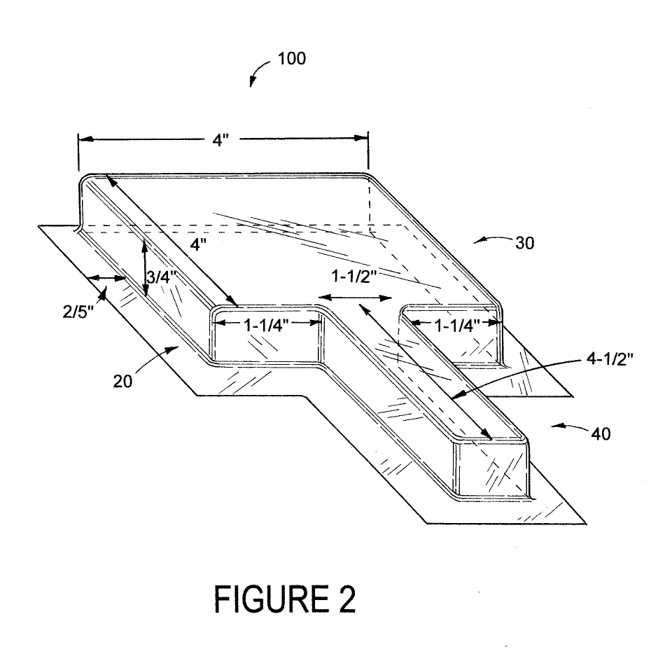

FIG. 2 is another drawing of the shower guard 100, this time showing possible dimensions, according to some embodiments. In one embodiment, the main portion 30 is a 4″ by 4″ square, extending ¾″ from the patient's body. Typically, the standard medical dressing used with a medical catheter is a 4″ by 4″ square dressing. In another embodiment, the main portion 30 is a 3″ by 3″ square, which is suitable for PICC catheters. Thus, the main portion 30 of the shower guard 100 is preferably sized so as to cover the medical dressing, allowing the adhesive portion 20 to surround the medical dressing without disturbing the dressing.

The extension portion 40 of the shower guard 100, extending from the main portion 30 and also ¾″ high, is designed to cover, but not touch, the catheter hardware comfortably. The base 20 extends about ⅖″ (1 centimeter) from the sides 34 and 44 of the main portion 30 and extension portion 40, and is preferably sized at approximately 1½″ by 4½″. In some embodiments, the inside of the shower guard 100 is treated with an antibacterial material. In other embodiments, the inside of the shower guard 100 is treated with a drying agent, such as a dessicant, for enhanced protection against water getting to the insertion site.

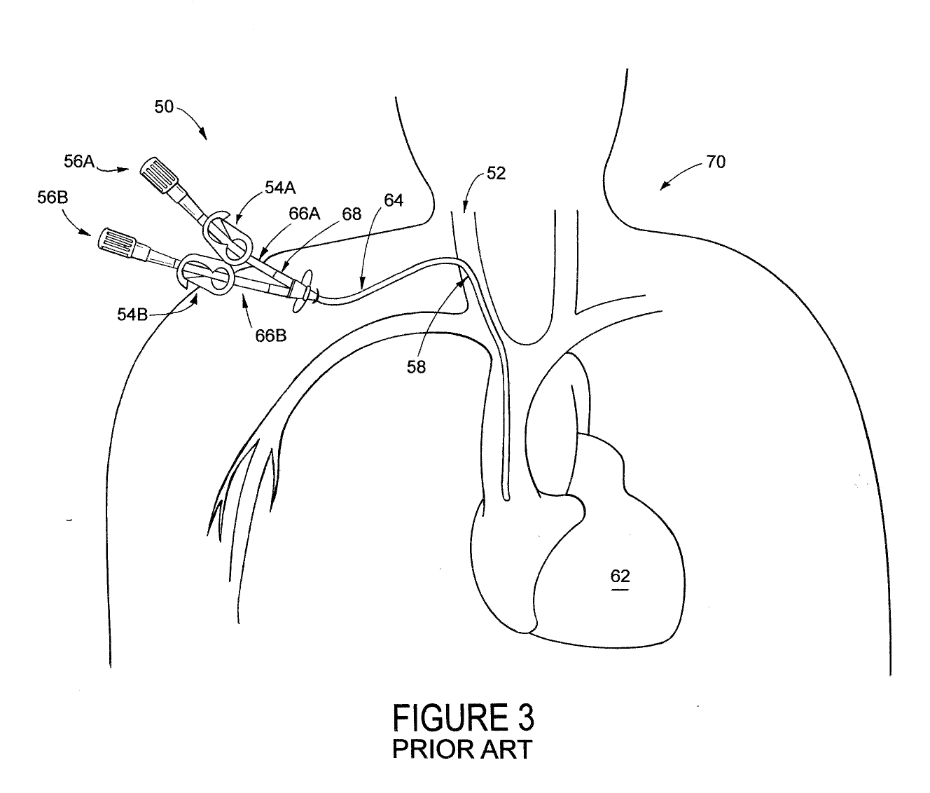

For patients with chronic kidney disease, the medical catheter 50 is used to connect a vein of the patient to the kidney dialysis machine. FIG. 3 is a cutaway diagram of a patient 70 connected to a medical catheter 50, according to the prior art. The medical catheter 50 consists of two clamps 54A, 56A (collectively, “clamps 54”), two caps 56A, 56B (collectively, “caps 56”), tubes 66A and 66B (collectively, “tubes 66”), a Y-connection 68, and an extension tube 64 to be inserted into the intrajugular vein 52 of the patient 70.

The cap 56A, the clamp 54A, and the tube 66A are known as a first lumen of the medical catheter 50, while the cap 56B, the clamp 56B, and the tube 66B are known as the second lumen of the medical catheter. Usually color-coded, one of the lumen is an arterial lumen (usually red), which transfers blood from the patient 70 to the dialysis machine (not shown); the other lumen is a venous lumen (usually blue), which transfers filtered blood from the dialysis machine to the patient.

The medical catheter 50 is connected to the patient 70 at an insertion point 58, which is a cut through the skin surface. The insertion point 58 allows the extension tube 64 to be inserted well into the intrajugular vein 52 of the patient 70. Upon installation of the medical catheter 50, the insertion point 58 is sewn with stitches by the medical professional.

Because the patient 70 will regularly be connected to the kidney dialysis machine, the medical catheter 50 remains coupled to the patient. This means that the patient 70 leaves the medical facility with the medical catheter 50 intact following an initial dialysis treatment, then may return perhaps a day or two later for a subsequent treatment. This process repeats, possibly for a very long period of time. For a chemotherapy patient, the treatments may cease after a period of weeks, but since the patient condition is chronic, kidney dialysis treatments continue for life, and only ceases for those ESRD patients who are fortunate enough to obtain a kidney transplant.

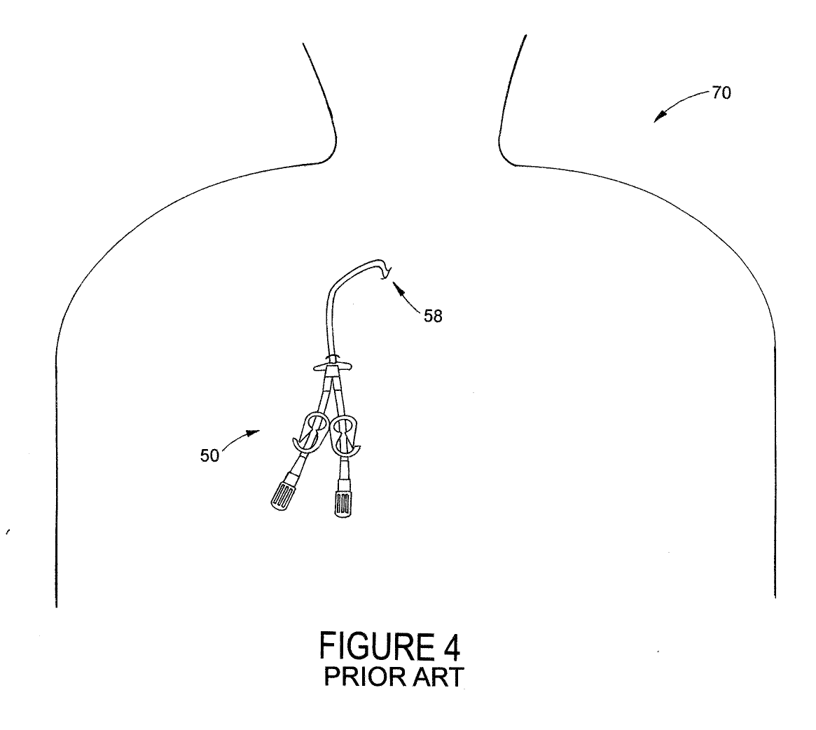

FIG. 4 shows the medical catheter 50 disposed at rest against the chest of the patient 70. The medical catheter 50 extends out of the insertion point 58 of the patient 70 and rests below the insertion point along the chest region of the patient. There are other insertion points for medical catheters 50 besides the intrajugular vein 52. The subclavian vein behind the clavicle is another insertion point. The medical catheter 50 may also be inserted into one of the veins of the arm or leg. The principles described herein are not meant to be limited merely to catheters inserted into the chest region, but may be applied to catheters inserted into any part of the human body.

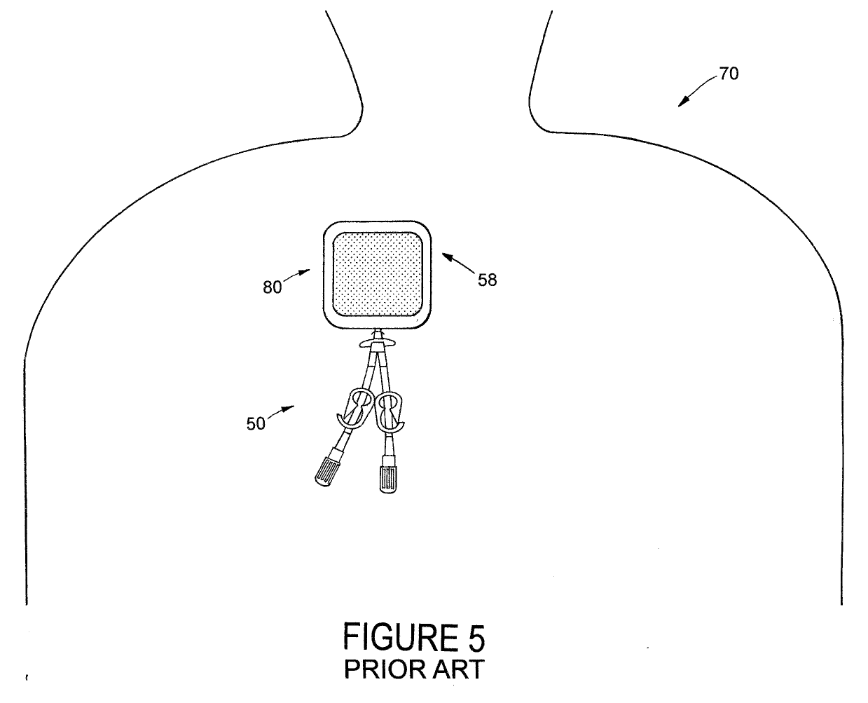

FIG. 5 shows how a medical dressing or bandage 80 is applied over the insertion point 58 of the medical catheter 50, according to the prior art. The medical dressing 80 is securely (usually adhesively) fitted over the insertion point 58, so that the insertion point will stay dry and not be exposed to ambient air, thus mitigating chances of infection at the insertion point. By applying the medical bandage 80 to the patient at this point, the insertion point 58 will be exposed to neither air nor water. Once applied, the patient 70 generally is instructed to neither remove the medical bandage 80 nor get it wet. This means that the patient cannot shower while the medical catheter 50 is attached, and must even bath with extreme caution so as to avoid wetting the medical bandage 80.

The medical bandage 80 itself is generally composed of a gauze or gauze-like material that is applied directly to the insertion point 58. The material is woven in such a way that it ideally does not adhere to the wound (in this case, the insertion site 58), but nevertheless protects the otherwise exposed site from the ambient air, thus mitigating the chance of infection. The medical bandage 80 is also generally surrounded by an elastomeric material (with latex being one example), which surrounds the gauze-like material. The elastomeric material is generally waterproof or semi-waterproof, and thus protects the gauze-like material from getting wet, again to prevent infection. A surrounding surface of the elastomeric outerportion of the medical bandage contains an adhesive material that enables the medical bandage 80 to affix to the patient's body. Ideally, the adhesive material makes no contact with the wound, thus keeping the medical bandage from disturbing the wound site.

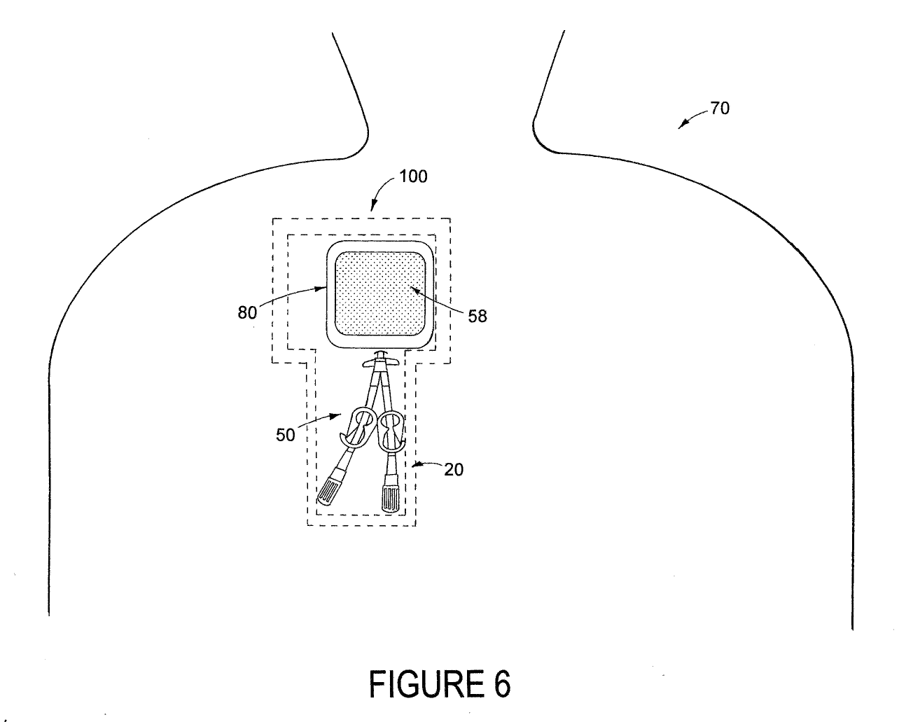

As shown in FIG. 5, while the medical bandage 80 covers the insertion site 58 and extension tube 54 of the medical catheter 50, the two lumen of the catheter remain exposed. FIG. 6 depicts a transparent view of the novel shower guard 100 being positioned over the medical catheter 50, according to some embodiments. The shower guard 100 is placed over both the medical bandage 80 and the exposed portion of the medical catheter 50. The adhesive portion 20 of the shower guard 100 is indicated with the dotted lines surrounding the medical bandage 80.

In some embodiments, the adhesive portion 20 of the shower guard 100 consists of an adhesive waterproof barrier, and includes a peel-away cover (not shown) for exposing the adhesive just before affixing the shower guard to the patient's skin. Ideally, the adhesive does not touch either the medical bandage 80 or the exposed portion of the medical catheter 50. Recall from FIG. 1 that the main portion 30 and extension portion 40 of the shower guard 100 are raised up from the surface of the patient's skin (e.g., ¾″). This means that, when affixed to the body of the patient 70, the shower guard 100 covers but does not touch either the medical bandage 80 or the medical catheter 50.

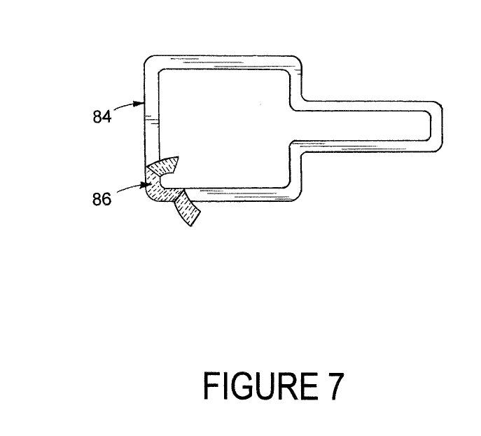

FIG. 7 is a side view of the novel shower guard 100, according to some embodiments. The adhesive portion 20 includes adhesive material 86, which is preferably evenly distributed over the edges of the adhesive portion 20. In some embodiments, the adhesive material 86 occupies approximately one centimeter of width surrounding the edge of the adhesive portion 20. The side view of FIG. 7 shows a peel-away cover 84 which is disposed over the adhesive portion 20 so as to prevent the adhesive material 86 from sticking to packaging and other surfaces prior to use of the shower guard 100.

In some embodiments, the shower guard 100 is intended as a single-use product and is disposable. While the shower guard 100 may be affixed to the body of the patient 70 by health care professionals, the shower guard is designed for the convenience of the patient and for use in the comfort of the patient's home. If affixing the shower guard 100 without assistance, prior to use, the patient 70 is preferably standing or sitting in front of a mirror so that she can clearly see her medical bandage 80 and medical catheter 50. The patient then removes the shower guard 100 from its packaging, removes the peelaway cover 84 from the back of the shower guard 100, and affixes the exposed adhesive material 86 directly to her skin at the site of the incision. Preferably, the main portion 30 of the shower guard is disposed directly over the medical bandage 80, preferably without the adhesive portion 86 touching the bandage, and the extension portion 40 of the shower guard is disposed directly over the exposed part of the medical catheter 50 (e.g., the lumens).

Once affixed to the body, the patient 70 may take a shower because the bandage 80 and catheter 50 are protected from exposure to the water, and thus risk of infection is avoided. After the patient is done showering and has dried at least the upper part of her body, the shower guard 100 may be removed and disposed of.

The novel shower guard 100 is thus a significant improvement over the aforementioned prior art solutions. U.S. Pat. No. 5,417,668, issued to Setzer, et al. (hereinafter, “Setzer”), discloses a protective cover that is used with a catheter in which the cover may be removed from the body and reinserted at a later time. The cover is not intended, however, to be waterproof in any way, and thus will not protect the patient from infection if the site gets wet.

U.S. Pat. No. 5,415,642, issued to Shepherd (hereinafter, “Shepherd”) also discloses a catheter cover, which is described as “fluid-impermeable”. The Shepherd catheter cover is applied over the incision site by the medical nurse, with the external portion of the catheter being inserted through the cover. This allows the patient to shower without concern that the site will become infected. Because the medical catheter must be manipulated in order to use it, the catheter cover must be installed by a medical professional.

U.S. Pat. No. 6,832,611, issued to Altman (hereinafter, “Altman”), is another catheter protection shield intended to protect the incision during showering, bathing, or swimming. As with the Shepherd patent, the Altman patent requires that a trained medical person apply the catheter protection shield to the site of incision.

The above inventions are impractical because they either fail to protect the medical bandage and insertion site from getting wet (Selzer) or they require trained medical personnel to install them on the patient's body (Shepherd and Altman). Thus, the patient is still unable to take a shower while at home while wearing the medical catheter.

The Selzer patent is not waterproof and is not suitable to enable the patient to shower. While enabling the patient to shower, the Shepherd catheter cover is impractical for two reasons. First, the catheter cover is required to be applied to the patient by medical personnel. Thus, the patient is only able to take a shower at the medical facility, following insertion of the Shepherd catheter cover through the medical catheter by trained personnel.

Second, because it has been threaded through the medical catheter by the doctor or other medical staff, the catheter cover must remain on the patient, which may be quite a bit more uncomfortable, such as during sleep, than the standard medical dressing. So, even if designed to be worn for a significant period of time, the Shepherd catheter is too uncomfortable to be worn during sleep, for example, and is thus an impractical solution that potentially increases the discomfort of the patient.

The Altman patent, a “catheter protection shield” similarly must be installed by a medical professional, as the shield includes an “inner bag” through which the medical catheter is required to be inserted. As with Shepherd, the complexity of the Altman invention makes it impossible for the patient to safely use the shield without the assistance of medical personnel, and, in lieu of medical personnel, increases the likelihood that the insertion site and medical bandage will be compromised or damaged, increasing the risk of infection to the patient.

In summary, there does not exist a protective shower guard created for indwelling catheters that fits securely over the top of the existing medical bandage 80 and medical catheter 50. Other solutions are inadequate because they either require a medical professional to install them or, where the patient attempts to use them, there is a high risk that the medical bandage 50 and/or medical catheter 50 will be compromised, thus increasing the risk of infection.

The novel shower guard 100 is thus a simple, user-friendly, disposable latex-free device that fits over and on top of the catheter 50 and bandage 80. The shower guard 100 will cover the catheter 50 without touching, pulling, or otherwise agitating, the stitches that close the insertion site 58. Further, the shower guard 100 fits over and on top of the standard size medical dressing 80. In some embodiments, the shower guard 100 is available in different sizes. The shower guard 100 is waterproof and allows patients to shower freely without risk of infections. The shower guard 100 includes a peel-away base, which will stick to the skin and provide a waterproof barrier.

In some embodiments, the novel shower guard 100 improves the independence of any patient with a catheter. By being able to shower regularly without risk of infection, the patient is more comfortable and has more control. Although designed for in-home use, hospitals and other medical facilities may also realize significant benefits when making the shower guard 100 available to its patients, due to decreased rates of infection.

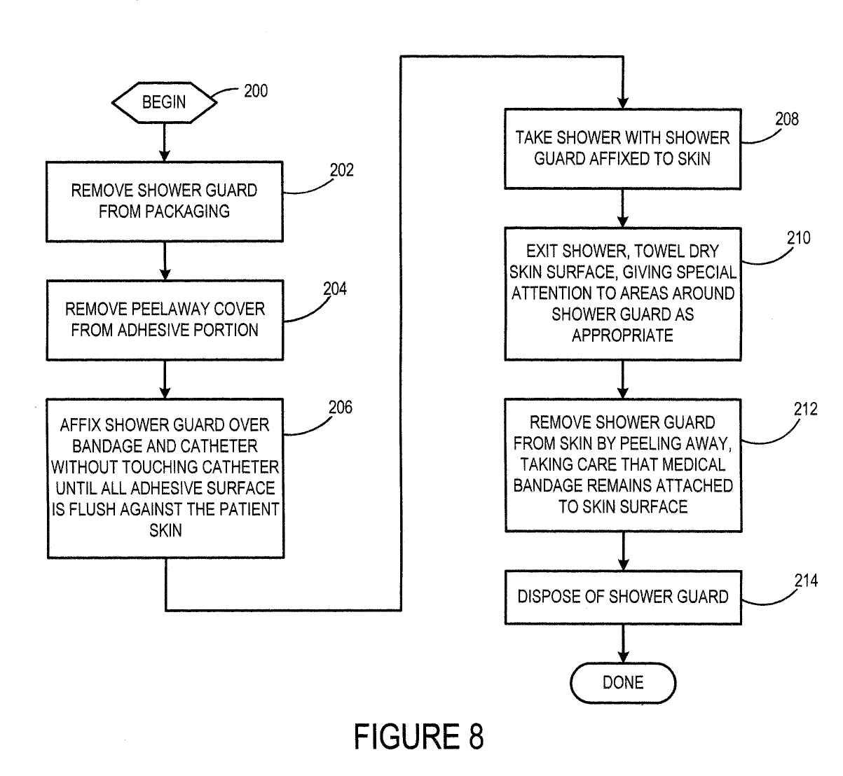

FIG. 8 is a flow diagram showing steps 200 that may be taken by a patient having a medical catheter in using the shower guard 100, according to some embodiments. The patient first removes the shower guard 100 from its packaging, if any (block 202). Next, the patient removes the peelaway cover 84 from the adhesive portion 20 of the shower guard 100 (block 204). This exposes the adhesive backing 86 of the shower guard 100, enabling the patient to affix the shower guard over the medical bandage and catheter (block 206). The patient preferably presses the shower guard 100 gently until the entire surface area of the adhesive backing 86 is flush against the patient's skin. At this point, the shower guard 100 is in place on the patient.

At this time, the patient is able to take a shower with the shower guard 100 securely affixed to her skin (block 208). Special caution may be taken where the shower produces a particularly strong stream of water. In the general case, however, no special precautions are necessary, as the shower guard 100 is designed for secure affixation to the skin surface. Once the bathing is completed, the patient may exit the shower and dry the surface of her skin (block 210). Special attention may be paid to the areas of the skin that are “above” the medical catheter and bandage. Where the medical catheter is inserted into the intrajugular vein, for example, the patient will want to ensure that the head, face, shoulders, and upper chest are dried before attempting to remove the shower guard 100 while standing, so as to mitigate the possibility of water dripping onto the medical bandage after removal.

Once the surface of the skin is dry, the patient removes the shower guard 100 by peeling the adhesive backing away from the surface of the skin (block 212). Although designed not to touch the medical bandage 80, the cautious patient will nevertheless ensure that the medical bandage is not also removed or partially removed during this step. Once removed from the patient's skin, the shower guard 100 may be disposed of (block 214). It is not recommended that the shower guard 100 be reused, as the adhesive backing will lose its efficacy over repeated use.

In some embodiments, the shower guard 100 is treated with specialized materials so as to further guard against water or infecting agents. An antibacterial coating may be applied to an inside surface of the shower guard. A dessicant such as calcium oxide or silica gel, which is an effective drying agent, may be attached to the inside surface, and may enhance the protective capacity of the shower guard 100.

While the application has been described with respect to a limited number of embodiments, those skilled in the art will appreciate numerous modifications and variations therefrom. It is intended that the appended claims cover all such modifications and variations as fall within the true spirit and scope of the invention.

I claim:

1. A shower guard for use by a patient having been fitted with a medical catheter, the shower guard comprising:
a main portion comprising a first surface and a plurality of sides disposed orthogonal to the first surface, the first surface and plurality of sides forming part of a rectangular cube, the main portion to be disposed over a medical bandage, wherein the medical bandage covers an insertion site of the medical catheter; and
an adhesive portion comprising a top surface and a bottom surface, the adhesive portion being disposed beneath the main portion, the adhesive portion being substantially parallel to the first surface, the bottom surface of the adhesive portion comprising an adhesive backing with a peelaway cover, wherein the peelaway cover, when removed, exposes the adhesive backing;
wherein the patient, without touching the medical catheter, affixes the adhesive backing to a skin surface such that the main portion is disposed over the medical bandage, allowing the patient to shower without getting the medical bandage wet.

2. The shower guard of claim 1, further comprising:
an extension portion comprising a second surface and a second plurality of sides, the second surface and second plurality of sides forming part of a second rectangular cube, the extension portion to be disposed over part of the indwelling medical catheter.

3. The shower guard of claim 2, wherein the first surface and the second surface comprise a continuous smooth surface.

4. The shower guard of claim 2, wherein the part of the indwelling medical catheter over which the extension portion is disposed comprises the lumen.

5. The shower guard of claim 1, wherein the rectangular cube comprises a dimension of 4″ by 4″ by ¾″.

6. The shower guard of claim 1, wherein the rectangular cube comprises a dimension of 3″ by 3″ by ¾″.

7. The shower guard of claim 1, wherein the rectangular cube is a size that enables the shower guard to be affixed to the skin of the patient without the adhesive backing touching the medical bandage.

8. The shower guard of claim 2, wherein the second rectangular cube comprises a dimension of 4½ by 1½″ by ¾″.

9. The shower guard of claim 2, wherein the main portion and the extension portion comprise a rigid waterproof material.

10. The shower guard of claim 1, wherein the adhesive portion comprises a waterproof material.

11. The shower guard of claim 1, wherein, upon removal from the skin of the patient, the medical bandage remains intact against the skin and covering the insertion site.

12. A shower guard comprising:
a main portion to be placed over a medical bandage of a patient, the medical bandage covering an insertion site for a medical catheter, the main portion being an open-ended rectangular cube, wherein two dimensions of the rectangular cube are slightly greater than the dimensions of the medical bandage; and
an extension portion to be placed over part of a medical catheter, the part of the medical catheter extending from beneath the medical bandage, the extension portion being a second open-ended rectangular cube, wherein two dimensions of the rectangular cube are slightly greater than the dimensions of the part of the medical catheter and the first and second rectangular cubes are coupled together;
wherein the patient, without touching the medical catheter, affixes the shower guard to the patient's skin such that the main portion is disposed over the medical dressing and the extension portion is disposed over the medical catheter, allowing the patient to shower without getting the medical bandage wet.

13. The shower guard of claim 12, further comprising:
an adhesive portion comprising a top surface and a bottom surface, the adhesive portion being orthogonally coupled to both the main portion and the extension portion, the bottom surface of the adhesive portion comprising an adhesive backing with a peelaway cover, wherein the peelaway cover, when removed, exposes the adhesive backing;
wherein the patient attaches the shower guard to the patient's skin using the adhesive portion.

14. The shower guard of claim 12, wherein the part of the medical catheter over which the extension portion is disposed comprises:
a first lumen comprising a first cap, a first clamp, and a first tube;
a second lumen comprising a second cap, a second clamp, and a second tube;
wherein the first tube and the second tube are joined by a y-connection.

15. The shower guard of claim 12, wherein the rectangular cube comprises a dimension of 4″ by 4″ by ¾″.

16. The shower guard of claim 12, wherein the rectangular cube comprises a dimension of 3″ by 3″ by ¾″.

17. The shower guard of claim 12, wherein, upon removal from the skin of the patient, the medical bandage remains intact against the skin and covering the insertion site.

18. The shower guard of claim 12, wherein an inside surface of the main portion and extension portion are treated with a dessicant.

19. A method of using a shower guard by a patient having a medical catheter, the method comprising:
removing, by the patient, a peelaway cover from an adhesive portion of the shower guard, wherein the removal exposes adhesive backing;
affixing, by the patient, the shower guard against a skin surface of the patient, wherein the shower guard, once affixed, is disposed over a bandage covering an insertion site of the medical catheter, and the patient does not touch the medical catheter during affixation;
showering, by the patient, with the shower guard intact against the skin surface;
removing, by the patient, the shower guard from the skin surface, wherein the patient does not touch the medical catheter and does not remove the bandage; and
disposing of the shower guard.

20. The method of claim 17, further comprising:
substantially drying, by the patient, the surface of skin surrounding the shower guard before removing the shower guard.
